(19) United States
(12) Patent Application Publication
(10) Pub. No.: US 2018/0235499 A1
(43) Pub. Date: Aug. 23, 2018

(54) METHOD FOR MEASURING AN ELECTROPHYSIOLOGICAL PARAMETER BY MEANS OF A CAPACITIVE ELECTRODE SENSOR OF CONTROLLED CAPACITANCE

(71) Applicant: Bioserenity, Paris (FR)

(72) Inventors: Sylvain Zorman, Paris (FR); Pierre Prot, Paris (FR); Pierre-Yves Frouin, Paris (FR)

(73) Assignee: Bioserenity, Paris (FR)

(21) Appl. No.: 15/751,661

(22) PCT Filed: Aug. 10, 2016
(86) PCT No.: PCT/EP2016/069019

§ 371 Date: Feb. 9, 2018

(30) Foreign Application Priority Data
Aug. 11, 2015 (FR) 1557676

(51) Int. Cl. A61B 5/0478; A61B 5/00; A61B 5/0492

(52) U.S. Cl. CPC A61B 5/0478; A61B 5/6814; A61B 2562/0214; A61B 5/0492; A61B 5/6805

(57) ABSTRACT

The invention relates to a sensor for measuring a physiological parameter of a subject, comprising:
a body (32) in an electrically insulating material, the body (32) comprising a base (31) and a plurality of protrusions (34) projecting from the base (31), and
a plurality of capacitive elements (37) in an electrically conductive material, embedded inside the body (32), each capacitive element (37) being positioned inside the body (32), at an end of a respective protrusion (34), so that when the ends of the protrusions (34) are in contact with the skin of the subject, the capacitive elements are at a predefined distance from the skin.

### FIELD OF THE INVENTION

The present invention relates to a sensor with capacitive electrodes, as well as to a device for measuring a physiological parameter of a subject, comprising such a sensor.

### STATE OF THE ART

Electrophysiology is the study of these physiological signals of electric nature. The most current measurements are the measurement of muscular activity with an electromyogram, the recording of the activity of the heart muscle with an electrocardiogram or the brain activity with an electroencephalogram.

These signals may be directly measured at the skin measurement area in a non-invasive way.

In order to continuously track the physiological condition of a user, the placement of conductive electrodes in contact with the skin measurement area is known. By the electric contact of the electrode at the skin measurement area, the variations of the electric potential resulting from the electrophysiological activity cause variation in the electric potential of the electrode. These variations are then directly recorded by an electronic circuit.

However, the operation of this type of sensor requires good electric contact with the skin measurement area which is generally obtained by using a gel or other conductive aqueous substance. Resorting to a conductive substance considerably degrades the ergonomics of the system for the subject, the stability of his/her characteristics overtime and the time for setting into place the electrodes in particular outside research or care centers.

The companies Cognionics, g.tec, emotiv and neuroelectrics have developed electrodes of the dry conductor type not requiring the addition of an electric contact gel between the skin measurement area and the electrode. These devices are described in documents U.S. Pat. No. 4,967,038-A, U.S. Pat. No. 8,326,396-B2, U.S. Pat. No. 8,644,904-B2, U.S. Pat. No. 8,548,554-B2.

However, said electrodes of the dry conductor type require electric contact with the skin measurement area and cause possible irritation of the skin. On the other hand, the weakness of the electric contact between the electrodes and the skin measurement area results in a high impedance and in degradation of the quality of the collected electrophysiological signals. For these systems, sweating is also a source of degradation of the quality of the signal.

In order to solve these limitations, so called capacitive electrodes not requiring any electric contact have been proposed.

Document GB 2,353,594-A describes said capacitive electrodes for electrophysiological measurements. But the absence of a suitable geometry does not give the possibility of guaranteeing a repeated and stable distance with the skin measurement area, notably in the areas with strong capillarity such as the scalp. The effective capacitance of the electrode is therefore subject to fluctuations which degrade the recorded signal.

Document US 2014/0171775 describes an intra-auricular capacitive electrode system. As this positioning of the electrode is not part of the standards of electrophysiology, such a measurement cannot generally be used within a medical or research environment.

### DISCUSSION OF THE INVENTION

An object of the invention is to propose a method for measuring an electrophysiological parameter by means of an integrated capacitive measurement device in a support allowing improved accuracy and ergonomics.

This object is achieved thanks to a sensor with capacitive electrodes for measuring a physiological parameter of a subject comprising an insulating body, and conductive capacitive elements.

The body consists of an electrically insulating material. It comprises a base and a plurality of protrusions projecting from the base. These protrusions give the possibility of crossing the capillary elements so that the ends of the protrusions are in direct mechanical contact with the measurement area.

Each of the capacitive elements consists of an electrically conducting material embedded inside the body. Each capacitive element is positioned inside the body, at an end of a respective protrusion, so that when the ends of the protrusions are in contact with the skin of the subject, the capacitive elements are at a predefined and constant distance from the skin.

Both of these characteristics give the possibility of placing the measurement elements, i.e. the capacitive elements, at a set distance from the measurement area in order to obtain a reproducible set capacitance and not affected by sweating.

The sensor may further have at least one of the following characteristics:

The body is formed of one single piece of material,

The body may be formed by molding the electrically insulating material directly on the capacitive elements.

The sensor comprises an electronic card extending inside the base of the body, and an electrically conductive wire connecting each capacitive element to the electronic card.

The body may be formed by molding the material around the capacitive elements, the electronic card and the electrically conductive wires. Thus, the whole of the components is encapsulated in the body, which gives the possibility of obtaining a device which may be immersed in water. This has an advantage in the case when the sensor is intended to be attached on a washable support, such as a piece of clothing for example.

The electronic card may be configured for generating a measurement signal of the physiological parameter depending on the electric potentials of the capacitive elements.

The sensor may also comprise a shielding layer positioned inside the body, and extending over a portion of the base. The shielding layer gives the possibility of reducing the sensitivity to electromagnetic perturbations not stemming from the measurement area.

The shielding layer may be positioned between the electronic card and the capacitive elements.

The sensor may further have a connector extending through the body in order to connect the electronic card to an external device for processing electric signals representative of an electric potential measured by the capacitive elements.

The invention also relates to a device for measuring a physiological parameter of a subject comprising:

a support capable of covering a portion of the body of the subject,

at least one sensor according to the preceding definition, the sensor being attached on the support so that when the subject is covered with the support, the support maintains the ends of the protrusions in contact with the skin of the subject.

The support gives the possibility of positioning the sensor simply and in a reproducible way. Additionally, the support allows application of a mechanical stress between the sensor and the measurement area. This mechanical stress gives the possibility of minimizing the perturbations associated with the movement of the sensor and ensures the mechanical contact of the sensor with the measurement area.

In an embodiment of the invention, the support is a piece of clothing able to cover the trunk of the subject in order to allow the recording of an electrocardiogram.

In another embodiment of the invention, the support is a piece of clothing able to cover the head of the subject in order to allow the recording of an electroencephalogram.

In another embodiment of the invention, the support is a piece of clothing able to cover the trunk of the subject in order to allow the recording of an electromyog ram.

In an embodiment of the invention, the device comprises a reference sensor and one or several measurement sensor(s). This gives the possibility of conducting so called differential measurements by using a so called reference electrode.

The invention further relates to a method for measuring a physiological parameter of a subject, by means of a measurement device according to the preceding definition, comprising a step of:

obtaining a reference signal by means of the reference sensor,

obtaining a measurement signal by means of the measurement sensor(s), and

obtaining a signal representative of the physiological parameter by subtracting the reference signal from the measurement signal.

In an embodiment of the invention, the method may also comprise a step of:

applying a corrective filter on the signal representative of the physiological parameter, the corrective filter increasing the relative amplitude of certain frequency components of the signal relatively to other frequency components.

Indeed, as explained below, the capacitive elements act like a high-pass filter. This filter modifies the signal which may be considered as a nuisance. The application of an adapted corrective filter (described below) gives the possibility of finding a remedy to this defect by correcting a posteriori the modifications of the frequency spectrum in order to obtain a more representative signal of the variations of the electric potential of the measurement area.

### PRESENTATION OF THE DRAWINGS

Other characteristics and advantages will further emerge from the following description, which is purely illustrative and non-limiting and should be read with reference to the appended figures.

FIG. 1 schematically illustrates an example of a device for measuring an electrophysiological parameter according to a first embodiment of the invention.

FIGS. 2A and 2B schematically illustrate, another example of a device for measuring an electrophysiological parameter compliant with a second embodiment of the invention.

FIG. 3 schematically illustrates, in a bottom view, a sensor with capacitive electrodes compliant with an embodiment of the invention.

FIG. 4 schematically illustrates in a sectional view, the sensor with capacitive electrodes of FIG. 3.

FIG. 5 schematically illustrates in a top view, the sensor with capacitive electrodes of FIG. 3.

FIG. 6A schematically illustrates, an example of an electronic circuit of a sensor with capacitive electrodes as well as of outer elements of the sensor with capacitive electrodes.

FIG. 6B schematically illustrates another example of an electronic circuit of a sensor with capacitive electrodes comprising a shielding system as well as elements outside the sensor with capacitive electrodes.

### DETAILED DESCRIPTION OF AN EMBODIMENT

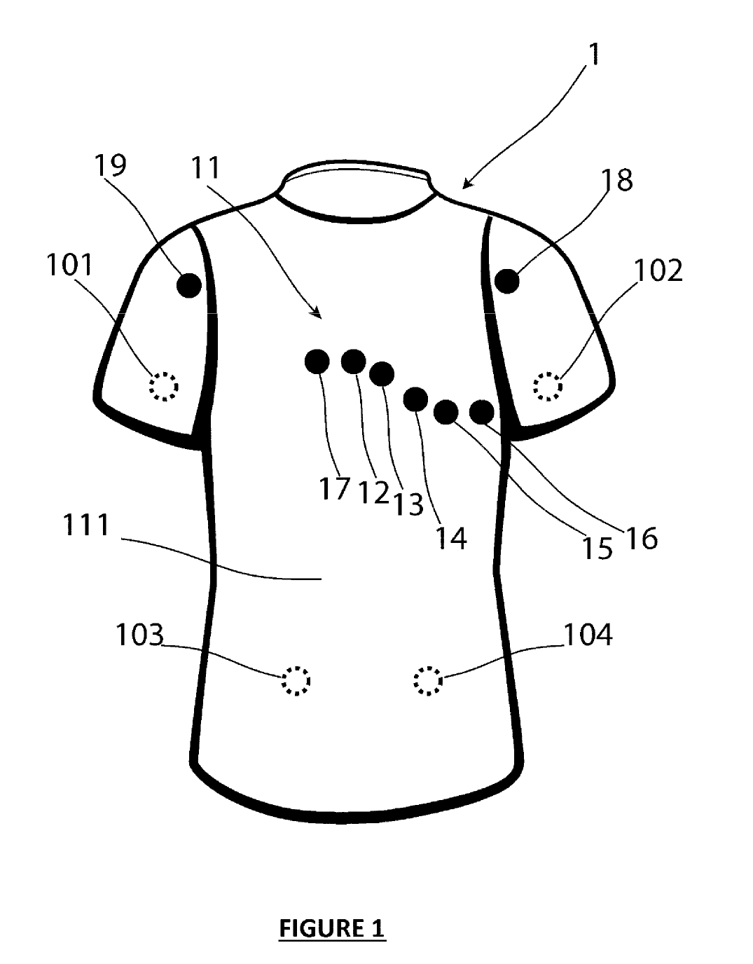

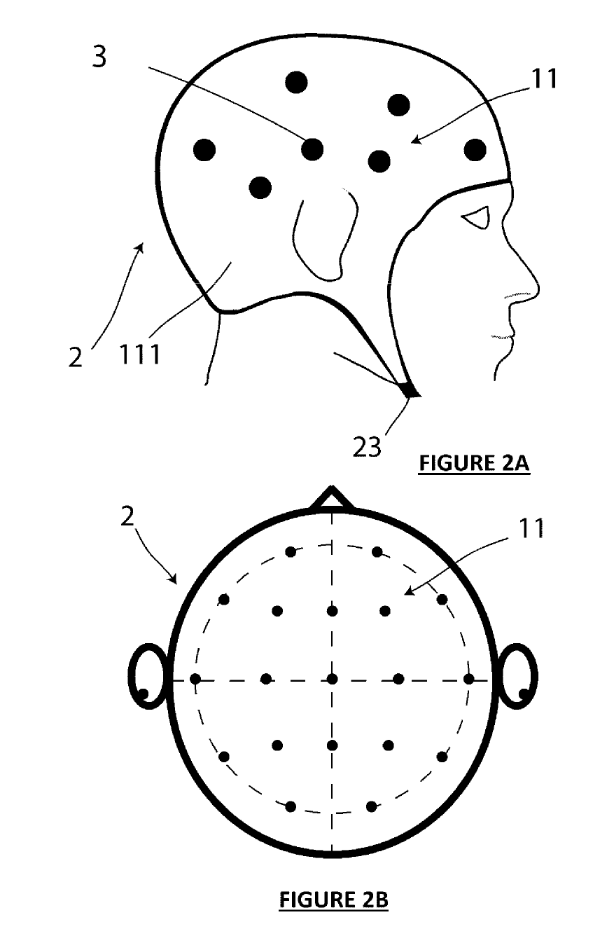

In FIGS. 1 and 2, the illustrated device for measuring electrophysiological signals comprises a plurality of sensors with capacitive electrodes 11 attached on a support 111 in order to track at least one electrophysiological parameter of a subject, for example an electromyogram or an electroencephalogram or an electrocardiogram.

The support 111 appears as a piece of clothing, such as a t-shirt or a cap, able to cover the measurement area.

The support 111 of the sensors with capacitive electrodes 11 has mechanical properties and a backing allowing application of a mechanical stress at the sensors with capacitive electrodes 11 improving the mechanical contact between the tip 33 of the protrusions 34 and the skin measurement area of the scalp 40.

In the embodiment illustrated in FIG. 1, the support of the sensors with capacitive electrodes is a t-shirt surrounding the chest.

In the embodiment illustrated in FIG. 1, the positioning of the sensors with capacitive electrodes from 13 to 19 allows recording of the heart electric activity and the sensors with capacitive electrodes 101 to 104 of the electric activity of muscles at the arms and at the abdomen.

The position of the sensors with capacitive electrodes 11 is predefined so that the putting on of the measurement device by the user causes predefined and reproducible positioning of the sensors with capacitive electrodes 11 at locations of the body allowing measurement of the electrophysiological parameter(s) of interest.

In a particular embodiment, illustrated in FIG. 2A, the device for measuring a physiological parameter is an electroencephalogram helmet 2.

In a particular embodiment, the positions of the sensors with capacitive electrodes in the cap 111 follow a well known mounting of type 10-20, like in the embodiment illustrated in FIG. 2B.

In a particular embodiment, a chin-strap 23 may be comprised in said electroencephalogram helmet 2 so as to increase the mechanical constraints on the sensors with capacitive electrodes at the scalp in order to improve the mechanical contact between the tips 33 of the protrusions 34 and the skin measurement area 40.

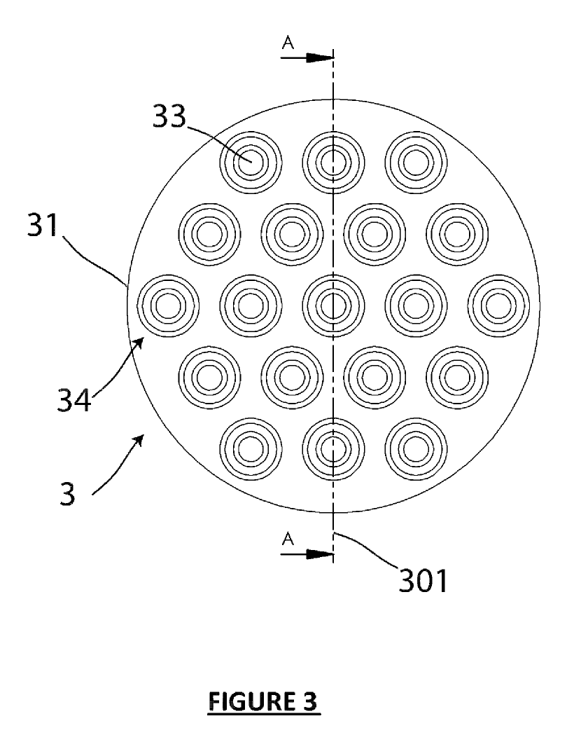

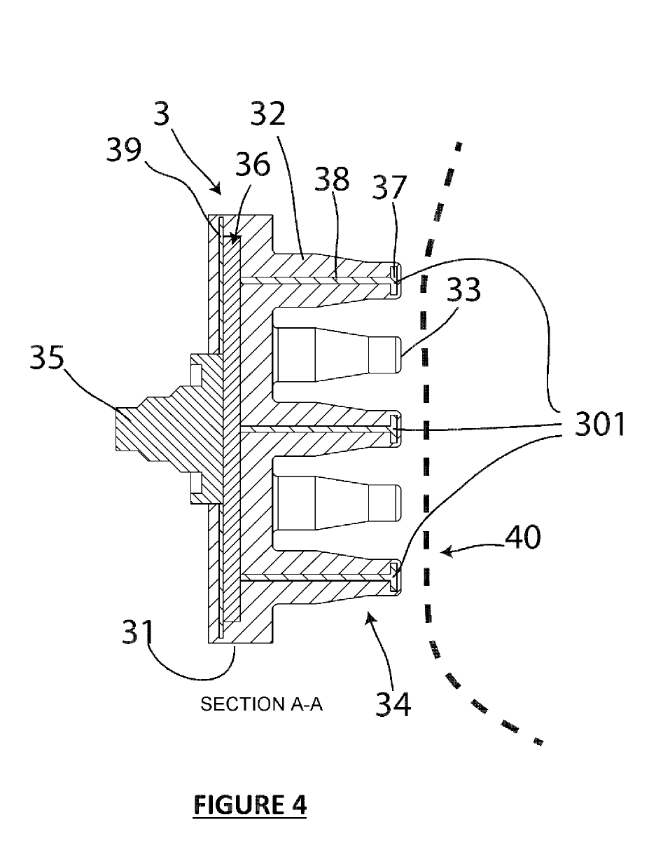

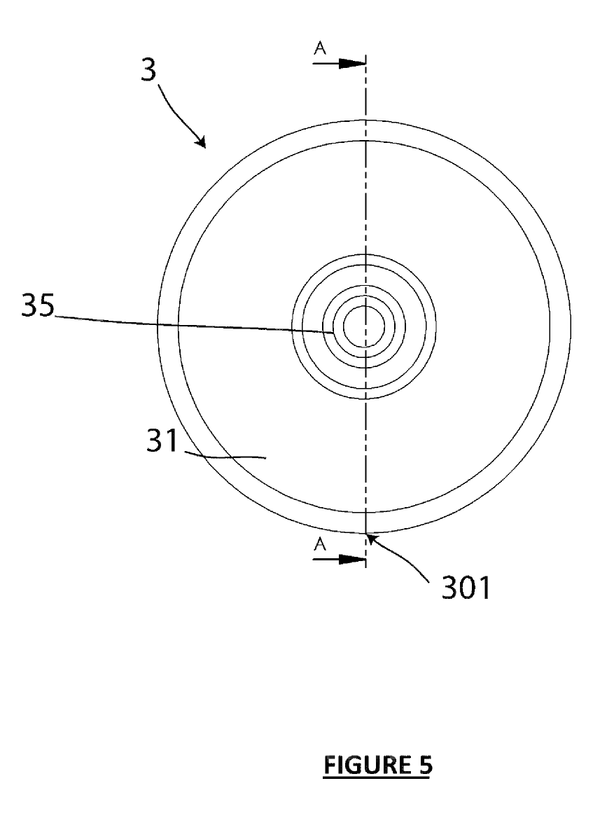

FIGS. 3 and 4 illustrate an embodiment of a sensor with capacitive electrodes 3.

In this embodiment, the sensor with capacitive electrodes 3 comprises a body 32 in an electrically insulating material. The body comprises a planar base 31 from 0.5 cm to 3 cm and a plurality of protrusions 34 extending in projection from the base 31. The body 32 is formed in a unique single piece of material.

The sensor with capacitive electrodes 3 further comprises a plurality of capacitive elements 301 in an electrically conductive material. Each capacitive element 301 is embedded inside the body 32, at the end of a protrusion 34, so that when the ends of the protrusions 34 are positioned in contact with the skin of the subject 40, the capacitive elements 37 extend to a predefined distance from the skin while forming a capacitor with the measurement area 40.

An electronic card 36 extends inside the base 31 of the body 32. Each capacitive element 37 is connected through a wire 38 to the electronic card 36.

A connector 35 extends through the body 32 for connecting the electronic card 36 to an external recording or physiological signal processing device.

The body 32 is preferably formed in a single piece of material, by molding around the capacitive elements 37, the electronic card 36 and the wires 38.

The protrusions 34 are distributed so that they are equidistant, according to a periodical or pseudo-periodical arrangement, depending on the selected embodiment. The number, the distance between the protrusions 34, the distribution of the protrusions 34 on the base 31 and the geometry of the protrusions 34 are optimized so that the protrusions 34 may cross the capillary thickness and in order to establish a direct mechanical contact with the skin measurement area of the subject.

Thus, depending on the subjects, the total absence or the very small number of capillary elements between the skin measurement area 40 and the tip 33 of the protrusions 34 resulting from the specificities of the embodiments shown here gives the possibility of making the distance between the skin measurement area and the capacitive element 37 repeatable and stable over time. This has the effect of making the value of the capacitance of the capacitor formed between the skin measurement area and the capacitive element 37 repeatable and stable over time, giving the possibility of significantly improving the quality of the signals within the context of electro-capacitive sensors.

The electric potential of each capacitive element 37 is particularly sensitive to variations in the electric field at the resulting measurement area 40 (see FIG. 4). Its electrical properties and it physical proximity to the skin measurement area 40 couple the potential of the capacitive element 37 at the tip of the protrusion 34 with the electric potential of the skin measurement area nearby 40.

The electrically insulating body 32, surrounds the whole of the elements of the capacitive electrode sensor except for the connector 35. The body 32 also imparts mechanical resistance properties to the sensor with capacitive electrodes.

In a particular embodiment, the protrusions 34 in number from 3 to 50, have an elongated shape and a diameter comprised between 0.5 mm and 3 mm so that they may cross the capillary areas and be in direct mechanical contact with the skin measurement area 40.

This mechanical contact with the skin measurement area of the ends 33 of said protrusions is constant during the measurement and ensures a constant and repeatable distance between the capacitive element 37 and the skin measurement area 40. With this characteristic, it is possible to cancel out the effects of skin sweating on the measurement of the electrophysiological potentials.

The thickness of the insulating material of the body 32 separating the capacitive element 37 from the skin measurement area 40 is comprised between 50 μm and 500 μm depending on the desired characteristics.

The value of the effective capacitance formed of the elements 37 and 40 depends on the geometry of the protrusions 34 and on the number of protrusions 34 per sensor with capacitive electrodes. More specifically, the capacitance depends on the diameter of a capacitive element 37, on the thickness of insulating material 32 between the elements 37 and the skin measurement area 40, on the electric permittivity of the insulating material 32 and on the number of protrusions 34 per sensor with capacitive electrodes 3. This value of the capacitance may be estimated by using the relationship C=ϵN a/d, with C the effective capacitance of the capacitor formed by the measurement area 40 and the element 37, ϵ is the permittivity of the insulating material of the body 32, N is the number of protrusions per sensor, a is the effective diameter of a capacitive element 37 and d the thickness of the insulating material between the capacitive element 37 and the skin measurement area 40. An alternative approach for estimating the capacitance may be achieved by using the finite elements method. In this approach, the skin measurement area may be modeled by a plane.

In the particular embodiment, the sensor comprises a shielding element 39 positioned inside the body 32 and extending over the width of the base 31.

The shielding element 39 associated with the electronic elements 42, 43 and 44 of the sensor with capacitive electrodes gives the possibility of reducing the parasitics generated by electromagnetic radiations produced by elements outside the measurement area. The shielding element 39 is maintained at a particular electric potential according to a technique consisting of using an operational amplifier 42 of which the non-inverting input is electrically connected to the electrically conducting elements 37. The inverting input is connected both to the shielding element 39 and to the output of the operational amplifier 42. This electronic circuit called a “follower” gives the possibility of maintaining the electric potential of the shielding element 39 at the same electric potential as the one of the capacitive elements 301. The shielding element 39 may then efficiently act for protecting the capacitive elements 301 from electromagnetic perturbations radiated by external apparatuses. The output of the amplifier 42 has the same electric potential as the one present on the capacitive elements 301, it therefore conveys a copy of the measured electrophysiological signal.

The sensor with capacitive electrodes comprises an electronic card 36 for amplifying and conditioning the electrophysiological signal copied at the output of the amplifier 42. This amplification and conditioning card comprises an amplifier 43 and resistors 44 and 444 as well as a capacitor 45, the electric properties of which give the possibility of determining the gain of the amplification. This gain, as well as the values of the resistors 44 and 444 and of the capacitor 45, are determined so that the level of the signal amplified in 43 is sufficient for being properly digitized by the ADC 47. Further, the resistor 444 and the capacitor 45 just upstream from the ADC 47 form a low-pass filter, the characteristics of which may be easily determined.

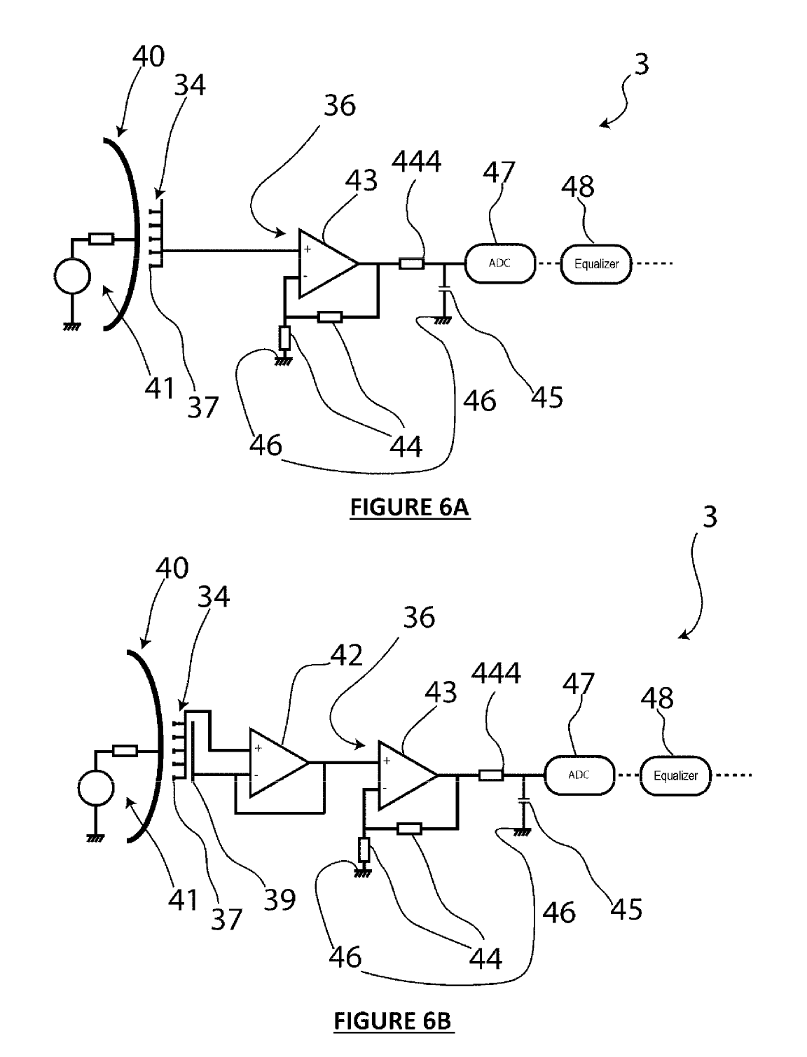

With reference to FIG. 6B, the transfer function of the capacitive element 37 associated with the operational amplifier 43, in the embodiment including a shielding 39 and the amplifier 42, expressed in a frequency space in polar coordinates is H_{capa}=(1+R_{AO}/Z_{capa})⁻¹, with R_{AO} being the effective input impedance of the elements 42 or 42 and 43 according to the embodiment and the impedance in polar coordinates Z_{capa} is defined by Z_{capa}=−i/ωC with i the imaginary unit, ω the angular frequency and C the electric capacitance of the capacitor, various estimation modes of which are described above.

In a particular embodiment, a second electronic circuit 48 connected to the capacitive electrode sensor 3 comprises a digital filter of which the transfer function H_{filter} is the reciprocal of the transfer function H_{capa} with H_{capa}×H_{filter}=1.

Given that the value of the capacitance of the capacitor, formed by the capacitive element 301 and the skin measurement area 40, is stable over time and repeatable, the transfer function of the sensor with capacitive electrodes is also stable over time and repeatable. Thus, the digital filter, of which the transfer function is predetermined, is always matched to the transfer function of the electrode 3, which guarantees good signal quality, stable over time and repeatable.

1. A sensor with capacitive electrodes for measuring a physiological parameter of a subject, comprising:
a body (32) in an electrically insulating material, the body (32) comprising a base (31) and a plurality of protrusions (34) projecting from the base (31), and
a plurality of capacitive elements (37) in an electrically conductive material, embedded inside the body (32), each capacitive element (37) being positioned inside the body (32), at an end of a respective protrusion (34), so that when the ends of the protrusions (34) are in contact with the skin of the subject, the capacitive elements are at a predefined distance from the skin.

2. The sensor according to claim 1, wherein the body (32) is formed in one single piece of material.

3. The sensor according to one of claims 1 and 2, comprising an electronic card (36) extending inside the base (31) of the body (32), and an electrically conductive wire (38) connecting each capacitive element (37) to the electronic card (36).

4. The sensor according to claims 1 and 2, wherein the capacitive elements (37), the electronic card (36) and the wires (38) are embedded in the material of the body (32).

5. The sensor according to one of claims 3 and 4, wherein the electronic card (36) is configured for generating a measurement signal of the physiological parameter depending on the electric potentials of the capacitive elements (37).

6. The sensor according to one of claims 3 to 5, comprising a shielding layer (39) positioned inside the body (32), and extending on a portion of the base (31).

7. The sensor according to claim 6, wherein the shielding layer (39) is positioned between the electronic card and the capacitive elements (37).

8. The sensor according to one of claims 3 to 7, comprising a connector (35) extending through the body (32) in order to connect the electronic card (36) to an external device for processing electric signals representative of an electric potential measured by the capacitive elements (37).

9. The sensor according to one of claims 1 to 8, wherein the body (32) is formed by molding the electrically insulating material directly on the capacitive elements (37).

10. A device for measuring a physiological parameter of a subject comprising:
a support (111) capable of covering a portion of the body of the subject,
at least one sensor according to one of claims 1 to 9, the sensor being attached on the support (111) so that when the subject is covered with the support (111), the support (111) maintains the ends of the protrusions (34) in contact with the skin of the subject.

11. The device according to claim 10, wherein the support (1, 111) is a piece of clothing able to cover the trunk of the subject in order to allow the recording of an electrocardiogram.

12. The device according to claim 10, wherein the support (2, 111) is a piece of clothing able to cover the head of the subject in order to allow the recording of an electroencephalogram.

13. The device according to claim 10, wherein the support (1, 111) is a piece of clothing able to cover the trunk of the subject in order to allow the recording of an electromyogram.

14. The device according to one of claims 11 to 13, comprising a reference device and one or several measurement device(s).

15. A method for measuring a physiological parameter of a subject, by means of a measurement device according to claim 14, comprising a step of:
obtaining a reference signal by means of the reference sensor,
obtaining a measurement signal by means of the measurement sensor(s), and
obtaining a signal representative of the physiological parameter by subtracting the reference signal from the measurement signal.

16. The method according to claim 15, comprising a step of:
applying a corrective filter on the signal representative of the physiological parameter, the corrective filter increasing the relative amplitude of certain frequency components of the signal relatively to other frequency components.
